(19) United States
(12) Patent Application Publication
(10) Pub. No.: US 2014/0296747 A1
(43) Pub. Date: Oct. 2, 2014

(54) HEAD PAD, SYSTEM TO STOP SNORING, AND METHOD THEREFOR

(71) Applicant: Johannes Herrnsdorf, Herdecke (DE)

(72) Inventor: Johannes Herrnsdorf, Herdecke (DE)

(21) Appl. No.: 14/358,518

(22) PCT Filed: Nov. 9, 2012
(86) PCT No.: PCT/EP2012/072338

§ 371 Date: May 15, 2014

Related U.S. Application Data
(60) Provisional application No. 61/636,160, filed on Apr. 20, 2012.

(30) Foreign Application Priority Data
Nov. 16, 2011 (DE) 102011118614.3

(51) Int. Cl. A61F 5/56; A61B 7/04; A61B 5/11; A61B 5/00; A47G 9/10; A61B 7/00

(52) U.S. Cl. CPC A61F 5/56; A47G 9/10; A61B 7/003; A61B 5/11; A61B 5/4818; A61B 5/4836; A61B 7/04
USPC 600/586; 128/845

(57) ABSTRACT

The invention relates to a head support for stopping a snoring of a sleeping individual, comprising an arrangement of neighbored deforming elements for setting a height of the head support section by section, wherein the deforming elements are arranged to allow a tilting of a head resting on the head support in two directions perpendicular to each other.

### CROSS-REFERENCE TO RELATED APPLICATIONS

This application is a National Stage of International Application No. PCT/EP2012/072338, filed on Nov. 9, 2012, and published in German as WO 2013/072262 A1 on May 23, 2013. This application claims the benefit and priority of U.S. Provisional Application No. 61/636,160, filed on Apr. 20, 2012 and German Application No. 102011118614.3, filed on Nov. 16, 2011. The entire disclosures of the above applications are incorporated herein by reference.

### BACKGROUND

This section provides background information related to the present disclosure which is not necessarily prior art.

1. Technical Field

The present invention relates to head supports, in particular pillows which has a function to reduce a snoring of a sleeping individual.

2. Prior art

A high percentage of individuals snores while sleeping. Snoring reduces the oxygen intake of the body and is nowadays known for provoking various diseases. A sleeping individual, however, does not perceive its own snoring so that it cannot readily be stopped by the individual on its own.

From prior art, various approaches are known to stop snoring of a sleeping individual. Devices that move the sleeping individual when a snoring occurs or which motivate the individual to change its position on its own are preferred in contrast to means that are directly attached onto the body or are directly inserted into the body, as they are more tolerated by the individuals concerned.

From document DE 109 30 818 C1, a device for preventing snoring is known which has a pillow, a sound sensor for detecting snoring noises and a control unit which can be activated by the snoring noises. The control unit controls a position change of the head of the sleeping individual by moving the pillow. For that purpose there is provided an airbag divided into chambers in the pillow wherein the air pressure of the chambers can be controlled by the control unit in conjunction with at least one air pressure source and an air pressure reduction unit.

From document DE 20 121 693 U1, a device for preventing snoring is known having an actuating unit for a head support of a sleeping individual wherein the actuating unit can be controlled by a control unit wherein the head support is activated when a snoring occurs to transfer vibrations onto the head of the sleeping individual so that the sleeping individual stops snoring. These vibrations, however, disturb an important deep sleeping phase which is important for the recreation of the individual.

From document DE 101 28 095 C2, a pillow for preventing the occurrence of snoring is known wherein at least one pressure chamber filled with a medium produces continuous steplessly controllable intervallic movements of the pillow by changing its volume wherein no harmful electrical fields are generated in the pillow.

### SUMMARY OF THE INVENTION

It is an object of the present invention to provide a head support and a method for operating a head support to stop a snoring of an individual sleeping on the head support in an improved manner, particularly without interrupting a deep sleeping phase important for recreation.

This object has been achieved by the head support according to the teachings of the present disclosure, by the system for stopping snoring, and by the method for operating the head support.

According to a first aspect, a head support for stopping a snoring of a sleeping individual is provided. The head support comprises an arrangement of neighboring deforming elements for setting the height of the head support in sections, wherein the deforming elements are arranged to allow a tilting of a head resting on the head support in two directions perpendicular to each other.

The above head support is based on the discovery that a movement of the head support for stopping snoring should not be arbitrarily but depends largely on the position of the sleeping individual or of the head of the sleeping individual, respectively, and its orientation. Thus, the head support is provided with deforming elements which are actuated depending on the occurrence of a snoring noise, wherein the deforming elements are actuated so that a tilting movement of the head around at least two axes is caused. This allows a tilting movement depending on to an actual orientation of the head, wherein the respective tilting movement is carried out which has been found for the respective orientation of the head of the sleeping individual as the tilting movement which can stop a snoring of a sleeping individual most reliably. A tilting and turning of the head in two directions, respectively, allow to move the head in almost every position and orientation which is anatomically possible.

It can be provided that a number of first deforming elements neighboring along a row in a cross-direction and one or more support elements are provided wherein the one or more support elements extend along a longitudinal direction perpendicular to the cross-direction and are offset to the row of the first deforming elements neighboring along the cross-direction.

According to an embodiment, the one or more support elements may abut the first deforming elements neighboring along a cross-direction and offset in a longitudinal direction perpendicular to the cross-direction.

Alternatively, it may be provided that the one or more support elements may be offset to the row of the neighboring first support elements with respect to a longitudinal direction perpendicular to the cross-direction and are distanced therefrom, wherein the distance is selected so that if the neck of the sleeping individual rests on the one or more support elements, the resting area of the back of the head rests on the row of the first deforming elements.

Furthermore, the one or more support elements can be arranged on an edge of the head support extending along the cross-direction.

It may be provided a number of first deforming elements neighboring in a row along a cross-direction, wherein one or more second deforming elements are provided which are offset into a longitudinal direction perpendicular to the cross-direction and which abut the row of the first deforming elements neighbored along the cross-direction. The cross-direction corresponds to a direction substantially perpendicular to the extension of the backbone of an individual resting on the head support. If the individual turns his head while resting on the head support, the head will roll in cross-direction. If the head support is atop of a lying area in a use arrangement, the cross-direction corresponds to the direction of the width of the lying area.

Furthermore, the deforming elements can be actuated separately to set the height of a section of the head support.

In particular, microphones for the detection of a snoring noise can be arranged at two opposing sides of the arrangement of the deforming elements. Alternatively only one or more than two microphones for the detection of a snoring noise can also be provided.

Furthermore, the deforming elements may include chambers for filling with a medium, wherein in the chambers or in the connected areas such as the conducts to the chambers, pressure sensors for determining a pressure in the chamber can be arranged.

According to a further aspect, a system for stopping a snoring is provided, comprising:
the above head support, and
a control unit which is configured to:
   detect a snoring noise;

if a snoring noise has been detected, determine an actual orientation of a head resting on the head support, and
actuate/control the deforming elements so that the head is tilted depending on the detected orientation of the head.

By the above system, the movement of the head can be controlled depending on its orientation so that the head is moved in a suitable manner if a snoring occurs.

Furthermore, the control unit can be configured to analyze signal propagation times and/or signal amplitudes of signals detected by microphones and/or a distribution of a pressure signal detected by pressure sensors in the head support or sensor signals detected by piezo sensors, vibration sensors, rotational speed sensors or acceleration sensors, in the head support, for detecting the orientation of the head.

According to an embodiment, the control unit may be configured to perform a learning process to determine at least one preferred tilting movement for the head, wherein, when a snoring occurs, the deforming elements can be actuated so that a head resting on the head support is moved according to the preferred tilting movement.

Particularly, the control unit can be configured to store the preferred tilting movement in association with a user profile.

The control unit may be configured to operate neighbored deforming elements for tilting a head so that the heights of the head support in the sections associated to the respective deforming elements are changed in opposite directions.

According to a further aspect, a method for operating a head support, in particular the above head support, is provided comprising:
detecting whether a snoring noise is present;
if a snoring has been detected, determining an actual orientation of a head resting on the head support; and
actuating the deforming elements so that the head is tilted depending on the detected orientation of the head.

### BRIEF DESCRIPTION OF THE DRAWINGS

Preferred embodiments of the present invention are described in more detail in conjunction with the accompanying drawings.

The drawings described herein are for illustrative purposes only of selected embodiments and not all possible implementations, and are not intended to limit the scope of the present disclosure.

FIG. 1 shows a system with a head support which is controlled by means of a control unit to move the head of the individual concerned, if a snoring occurs;

FIG. 2 a cross-sectional view of the head support of the system of FIG. 1;

FIG. 3 a flowchart representing a method for operating the system of FIG. 1;

FIG. 4 shows a further embodiment of a system with a head support which is controlled by means of the control unit to move the head of the individual concerned if a snoring occurs;

FIG. 5 shows an on-top cross-sectional view of a further embodiment of a head support which comprises a neck support element; and

FIG. 6 shows a cross-sectional view of the head support of FIG. 5 along the line A-A.

Corresponding reference numerals indicate corresponding parts throughout the several views of the drawings.

### DETAILED DESCRIPTION OF THE PREFERRED EMBODIMENTS

Example embodiments will now be described more fully with reference to the accompanying drawings.

Elements of the same or similar function are referenced by the same reference signs.

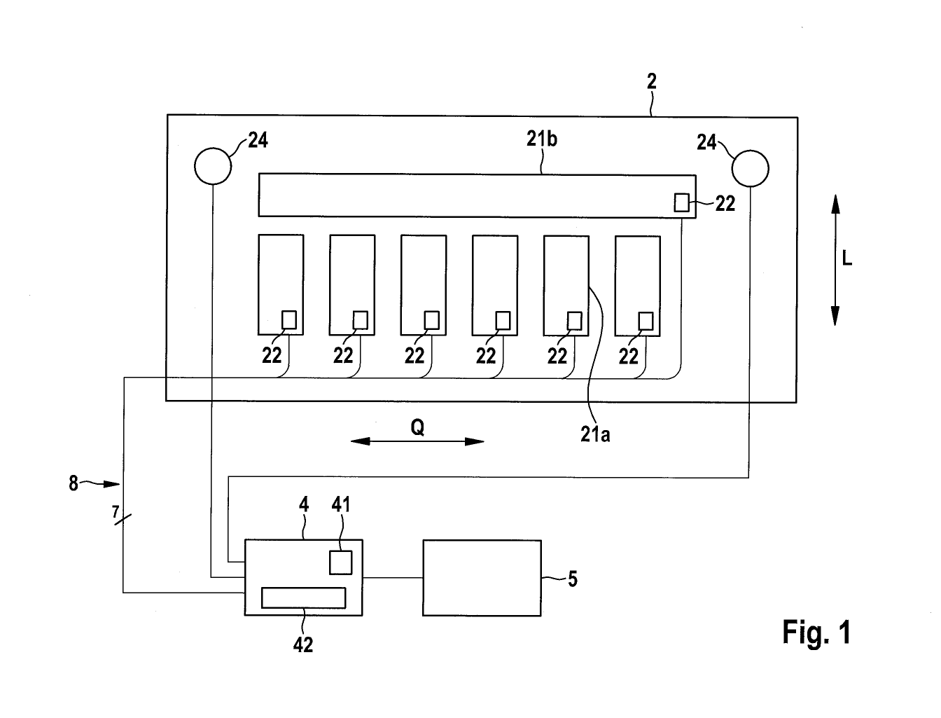

FIG. 1 schematically shows a representation of a system 1 for stopping a snoring. The system 1 comprises a head support 2 which may serve as active pillow for a sleeping individual or as active underlay for the head of the sleeping individual. Furthermore, the head support 2 is connected with a control unit 4 which controls the functions to be carried out with the head support 2. In an alternative embodiment, the control unit 4 may also be integrated in the head support.

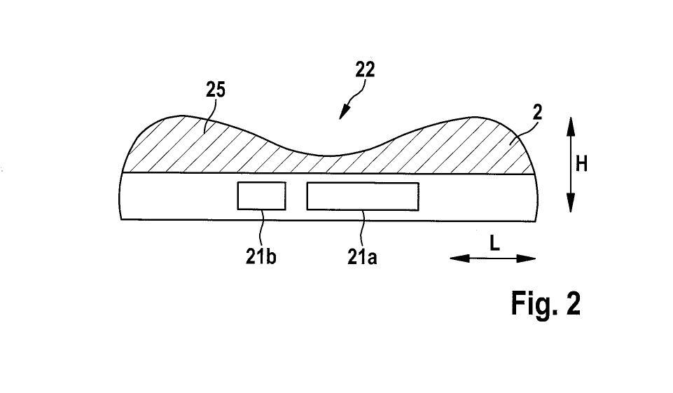

The head support 2 has a substantially rectangular shape with a cross-direction Q and the longitudinal direction L. The head support 2 shall be arranged in a bed so that the cross-direction Q corresponds to the width direction of the bed or the lying area, respectively, and the longitudinal direction L corresponds to the longitudinal direction of the bed or the lying area, respectively. As is further shown in conjunction with the side view of the head support 2 of FIG. 2, the cross-section of the head support 2 with respect to the cross-direction Q is provided with a varying height in the height direction H. Particularly, the head support 2 has a recess 22 to define the position of the head of a sleeping individual with respect to the longitudinal direction L. This is advantageous as the specific tilting movement of the head in a longitudinal direction L is facilitated.

The head support 2 comprises a number of deforming elements 21. The deforming elements 21 can be actuated in an appropriate manner to deform, in particular to elevate or to lower the head support 2 at the position of the respective deforming element 21.

The deforming elements 21 can, as shown in the present embodiment, be provided as gas-filled chambers made of a flexible material, particularly as air chambers which can be filled with a fluid, particularly a gas, such as air, or a liquid, such as a gel or water. The amount of the liquid which is put into the chambers determines the deformation of the head support 2, particularly the height of the head support 2 at the position associated with the respective deforming element 21.

A reservoir containing fluid which is to be put into the chambers can be provided in the interior of the head support 2 or separately therefrom. In the present embodiment, an external air pump 5 is provided which intakes environmental air and supplies it under an increased pressure. Alternatively, an air pressure tank can be provided instead or additionally to the air pump 5, wherein the air pressure tank can be filled or replaced at non-sleeping times to avoid operational noise of the air pump 5 during sleeping times.

Alternative configurations of the deforming elements 21 are possible, such as e.g. electromechanical actuators and the like as long as they can cause a height change of a section of the head support 2.

A head support 2 can furthermore be provided with a cushion material, such as a foam material and the like, so that the deforming elements 21 are separated from the head of the sleeping individual by at least a cushion or a damping layer, respectively. Hence, the comfort can be improved for use of the head support 2.

The number and arrangement of the deforming elements 21 is selected so that the tilting or turning of a head resting on the head support 2, respectively, with respect to at least one direction, preferably to the cross-direction Q. For this purpose, a row of similar first deforming elements 21a are neighbored along a cross-direction Q. The first deforming elements 21a can have a rectangular cross-section with respect to the height direction of the head support 2, the rectangular cross-section having a shorter side along the cross-direction Q.

Also a polygonal and/or alveolar configuration of the first deforming elements 21a can be provided. Furthermore, the first deforming elements 21a can be arranged along a curved line. This can be achieved by providing the first deforming elements 21a with a cross-section differing from a rectangular cross-section with respect to the height direction H, e.g. a trapezoidal cross-section or an annular segment cross-section.

By selectively deforming the deforming elements 21, a turning or tilting movement of the resting head in the cross-section Q can occur. In particular, this can be achieved in that two neighbored first deforming elements 21a above which the resting area of the head resting thereon extends, can be deformed differently; in case of chambers as deforming elements 21 this is particularly achieved in that one of the two neighbored first deforming elements 21a is filled and another one of the two neighbored first deforming elements 21a is emptied. If the resting area of the head extends over more than two deforming elements 21, it may of course be provided respective deformations of more than two deforming elements 21 to accomplish the respective tilting of the head.

To achieve a tilting of the head in a longitudinal direction L perpendicular to the cross-direction Q, one or more second deforming elements 21b can be provided which abut the row of the first deforming elements 21a arranged along the cross-direction Q with respect to the longitudinal direction L, and which may have substantially the same width as the row of the first deforming elements 21a. In the shown embodiment, the second deforming element 21b extends over the whole width of the arrangement of the first deforming elements 21a.

There are known various variants of the arrangement of deforming elements 21, however, these should be arranged with respect to each other so that, by respective actuating of one or more deforming elements 21, the height of the head support can be set or altered, respectively, in the section in which the deforming elements are positioned. Particularly, the purpose of the arrangement of the deforming elements 21 is that when the head rests in an active area of the head support 2 in which the deforming elements 21 are placed, the respective heights of at least those deforming elements 21 arranged beneath the resting area of the head can be changed so that the head can be tilted at least along the cross-direction Q preferably also in the longitudinal direction L.

In the above example, the tilting can be achieved in that in the section in which the head rests on the deforming elements 21, the deforming elements 21 are actuated so that the height of the head support 2 increases, while in the direction towards which the head shall be tilted, the respective deforming element 21 is actuated so that the head support 2 has a lower height. So, a tilting momentum is applied onto the head, the amount of which substantially depends on the height difference to which the head is exposed due to the different setting of the heights of the neighbored deforming elements 21.

In the above embodiment, the tilting of the head along the longitudinal direction L can be achieved by a respective setting of the second deforming element 21b. To support a tilting movement along a longitudinal direction L, the deforming elements 21 arranged along the cross-direction Q can additionally be actuated in an opposing manner so that a height change, i.e. either an elevation or a lowering of the height of the head support 2, which acts uniformly and over the whole width of the arrangement in a cross direction Q opposing the direction of the height change of the second deforming element 21b, can be caused.

The head support 2 is further provided with one or more microphones 24, which allows to detect a snoring noise of an individual sleeping on the head support 2. To reliably detect the snoring noise, preferably at least two microphones 24 are arranged at the ends of the head support 2 along the cross-section Q. Hence, the snoring noise of an individual who is resting on its side can be detected reliably.

The snoring noise can also be detected by a pressure sensor provided in the head support 2 or by an external vibration sensor instead of the microphones 24. Furthermore, the head support 2 can be provided with a sensitive lamination, such as a lamination with resistive, capacitive or piezo-resistive measurement characteristics to detect vibrations caused by the snoring noises. Alternatively, a bed frame on which the head support 2 is placed can be provided with a respective sensing means to detect vibrations caused by a snoring.

The deforming elements 21 are actuated/controlled by the external control unit 4. The control unit 4 is connected both with the deforming elements 21 and with the microphones 24. If at least one of the microphones 24 detects a snoring noise, a respective electrical signal is analyzed by the control unit 4 to identify a snoring or to determine whether or not the detected noise is a snoring noise, respectively. In a microcontroller of the control unit 4, an appropriate sound recognition algorithm can be implemented which can include a function of a speech recognition algorithm in principle.

If a snoring noise has been determined, the control unit 4 actuates the deforming elements 21 in an appropriate manner. In the present embodiment, the control unit 4 is connected with the deforming elements 21 formed as air chambers, via air conducts 8. So, the provision of electrical connections to the head support 2 can be avoided. Of course, also electromechanical actuators as deforming elements 21 can be provided which can be actuated by means of electrical connections.

The control unit 4 is connected with the deforming elements 21 formed as air chambers by means of the respective air conducts 8. Furthermore, the control unit 4 is connected with the air pump 5 for a supply with pressured air. Apart from the microcontroller 41, the control unit 4 has solenoid valves 42 which may control the air supply to the individual air chambers. The solenoid valves 42 allow to actuate each of the air chambers individually, so that they can be filled with pressured air or so that pressured air can be released therefrom.

Basically, the control unit 4 serves to actuate the deforming elements 21 for moving the head of a sleeping individual resting thereon as soon as a snoring has been detected by the microphones 24.

It has been shown in experiments that, after a snoring occurred, a random movement of the head only results in a stopping of the snoring with a very low likelihood. Moreover, it has been found that a specific tilting movement of the head results in a stopping of the snoring of a sleeping individual with a high likelihood. Particularly, tilting movements of the head are, however, necessary depending on the orientation of the head of the sleeping individual, i.e. depending on whether the sleeping individual sleeps in a side or supine position, to have high chances stop the snoring. In addition, the manner of the tilting movement is individually different for each snoring individual, and an interruption of the snoring can occur at a tilting movement in a cross-direction Q or in a longitudinal direction L or in a direction including both the cross-direction Q and the longitudinal direction L.

The control unit 4 provides, depending on the orientation of a head of a sleeping individual on the head support 2, i.e. depending on whether the head is in a side orientation, a supine (back) orientation or in an intermediate orientation between the side orientation and the supine orientation to perform the specific tilting movement as soon as a snoring noise has been detected. As described above, the tilting movement has to be different depending on the orientation of the head for an efficient effect, i.e. whether the sleeping individual is in a side orientation or in a supine orientation.

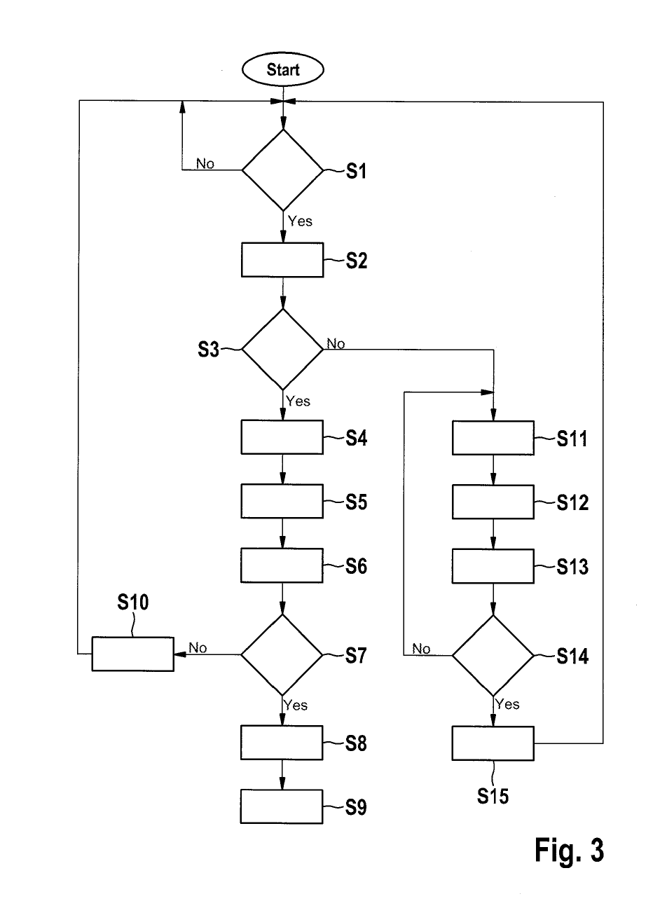

The control unit 4 can carry out a method for stopping a snoring, as it is shown in the flowchart of FIG. 3. Thereto, the control unit 4 may also provide to start a learning process on occurrence of a snoring, if the sleeping individual is unknown, to determine which tilting movement results in a stopping of the snoring for the respective individual.

If in a branching step S1 a snoring has been detected (alternative: yes), the method is continued with a succeeding step S2. Otherwise (alternative: no), it is jumped back to step S1.

The tilting movement of the head of the sleeping individual to be carried out can be different depending on the orientation of the head (side orientation or supine orientation) so that it is determined in step S2 in which is the actual orientation of the head of the sleeping individual. Furthermore, it is necessary to determine the relative position of the head on the head support 2 to determine the respective deforming elements 21 needed for carrying out the tilting movement of the head so that they can be actuated accordingly.

To detect the position of the head of the sleeping individual, the deforming elements 21 formed as air chambers can be provided with pressure sensors 22 so that the respective air chamber having a pressure higher than the pressure of the other air chamber can be recognized as the one air chamber which carries the main weight of the head. Alternative possibilities to determine the position of the head of the sleeping individual on the head support 2 can include the provision of capacitive or resistive pressure sensor arrays on the head support 2, particularly of sensor arrays which are built with conductive foams or the like.

Alternatively, a camera may be provided, in particular a camera which is sensitive to infrared and which is directed onto the head support 2 and which can determine the position of the head of the sleeping individual, i.e. the relative position of the head on the head support 2 by means of an appropriate image recognition technique.

Furthermore, it is necessary to provide an algorithm for determining the orientation of the head. The orientation of the head can be determined e.g. by determining the resting area of the head. In other words, as the resting area of the back head is smaller than the resting area of a side of the head, the orientation of the head can be determined by analyzing of pressure increases in the air chambers. For instance, it can be concluded a supine orientation, if an increase of pressure in one or two chambers is detected (with respect to the air chamber with the lowest air pressure exceeding a given pressure threshold), and can be concluded a supine orientation, if an increase in pressure in more than two chambers is detected (increase of pressure with respect to the air chamber with the lowest air pressure exceeding a given pressure threshold).

Alternatively or additionally, the orientation of the head can be determined by means of the microphones 24 which are arranged in the head support 2 and opposing each other with respect to the cross-direction Q. Based on the snoring noises detected by the microphones 24, the position and orientation of the head of a sleeping individual can be determined by means of an appropriate signal processing. Since in a supine position the nose as origin of the snoring noise is arranged substantially in the middle of the head, the position of the nose can be simply determined by propagation delay differences of the snoring noise to the microphones 24. Due to the symmetry of the head, the snoring noise is dampened on the way to the microphones 24. This damping depends merely from the distance of the nose to the respective microphone 24. If the head of the sleeping individual is in a side orientation, the signal amplitude of the microphone 24 directed to the backhead is strongly attenuated and a side orientation of the head can be determined by a comparison of the signal amplitudes of the signals detected by the microphones 24.

Substantially, side or supine orientations of the head of the sleeping individual can be distinguished by analyzing the damping of the acoustic snoring signal. If one of the microphones 24 detects a too strongly damped snoring signal, it can be concluded that the head of the sleeping individual rests on the side orientation. The respective microphone 24 is associated to the backhead. The presence of a too strongly damped snoring signal can be determined, e.g. by calculating the relation of the amplitudes of the electrical signals detected by the microphones 24 and by means of a threshold comparison, wherein the threshold value of the ratio of the signal amplitude is selected depending on the position of the head on the head support 2 to be able to consider the damping which is merely caused by the distance between the nose and the microphones 24.

It is possible, in a calibration process, to place a head which emits a snoring noise in a supine position at different positions of the head support 2 and to perform corresponding signal measurements, i.e. measurements of the propagation delay differences and the signal dampings by means of the microphones 24 and to repeat this process with different side orientations of the head at different positions on the head support 2. By storing the propagation delay differences and the signal amplitudes of electrical signals provided by the microphones 24 in conjunction with the associated position and orientation of the head, a reference table can be made which is stored in the control unit.

If a snoring noise occurs, the reference table can be accessed. For this purpose, the propagation delay difference and the ratio of the signal amplitudes of the electrical signals supplied by the microphones 24 are determined, and the actual position of the head of the sleeping individual and its orientation are determined by appropriate referencing. Thereto, the position and orientation from the reference table can be selected as the actual position and the orientation of the head at which a difference between the detected propagation delay difference and the propagation delay difference taken from the reference table and the difference between the detected ratio of the signal amplitudes and the ratio of the signal amplitudes taken from the reference table is minimum. Particularly, the method of the minimum squared error can be applied thereto.

In a succeeding branching step S3, it is checked whether a learning process for the head support 2 has been carried out before.

If positive (alternative: yes), a measurement pattern is retrieved from a memory 43 of the control unit 4 in step S4 depending on the orientation of the head detected before and, in step S5, the deforming elements 21 of the head support 2 are actuated correspondingly to cause the tilting movement of the head according to the movement pattern. The movement pattern indicates the direction of the tilting of the head. Additionally, the movement pattern can further indicate a velocity of the tilting of the head. The velocity of the tilting can be controlled by a corresponding actuation of the deforming elements 2, i.e. the deforming elements 21 are operated so that a faster or slower change of the height of the corresponding section of the head support 2 is caused.

After the movement of the head in step S5, a predetermined time period is waited in step S6, e.g. 30 seconds or 1 minute to determine whether the movement of the head of the sleeping individual has stopped its snoring.

If it is determined in a succeeding branching step S7 that the snoring continues (alternative: yes), a further movement pattern is retrieved from a memory 32 in step S8 and is executed in step S9. Thereafter, it is branched back to step S7, and the cycle is repeated if the snoring further occurs.

If no snoring is determined in the branching step S7 (alternative: no), a gradual movement of the head is carried out to bring the head back into a start position, and it is branched back to step 51.

When it is determined in step S3 that no learning process has been carried out before (alternative: no), the following tilting movements are carried out according to a given learning pattern by a corresponding actuation of the deforming elements 21.

The learning pattern can provide a sequence of tilting movements which are stored in the memory 43 appropriately.

For instance, the following sequence of tilting movements can be provided as learning pattern for a supine orientation of the head:
1. tilting movement in cross-direction Q to the left,
2. tilting movement in cross-direction Q to the right,
3. tilting movement in longitudinal direction L upwards,
4. tilting movement in longitudinal direction L downwards.

The following sequence of tilting movements can be provided for a side orientation:
1. tilting movement in longitudinal direction L upwards,
2. tilting movement in longitudinal direction L downwards,
3. tilting movement in cross-direction Q in direction to a supine orientation of the head,
4. tilting movement in cross-direction Q in direction opposing to the supine orientation.

The tilting movements can be provided as relative tilting movements of the head or can be provided as stop positions into which the head shall be moved/turned.

In a step S11, the first or the next movement pattern is retrieved from the memory, and in step S12, the head of the sleeping individual is moved correspondingly. In step S13, it is waited as described in step S6 for a given time period, such as a time period between 30 seconds and 1 minute, to determine whether the movement of the head of the sleeping person has caused an interruption of the snoring.

In the branching step S13, it is determined whether the snoring has stopped. If positive (alternative: yes), the lastly performed tilting movement (and optionally the tilting velocity) is stored as movement pattern in conjunction with the lastly determined orientation of the head in step S15. Otherwise, it is branched back to a step S11.

The learning process of the steps S11 to S14 is carried out as long as the movement of the head has been found at which the snoring of the sleeping individual has been stopped. Alternatively, the learning process can be continued with all tilting movements of the respective learning pattern to determine multiple tilting movements of the head by which a snoring of the sleeping individual can be interrupted.

After a preferred tilting movement for the head of a specific individual has been found by the learning process, the respective tilting movement can be stored in a personalized manner. An association to the respective individual can be carried out, e.g. by storing of an appropriate profile. By selecting of the specific individual, e.g. by means of a suitable input unit of the control unit 4, the previously learned tilting movements can be carried out in case a snoring occurs.

It is possible to learn and to store profiles for multiple individuals using the same head support 2. Alternatively, to the selection of the concerned individual by means of the input unit, the occurrence of a specific snoring noise can be associated to a specific individual by means of a simple speech or snoring recognition method. After this association has been made, the corresponding profile associated to the snoring noise is selected and the tilting movements stored therein are carried out in the above-described manner.

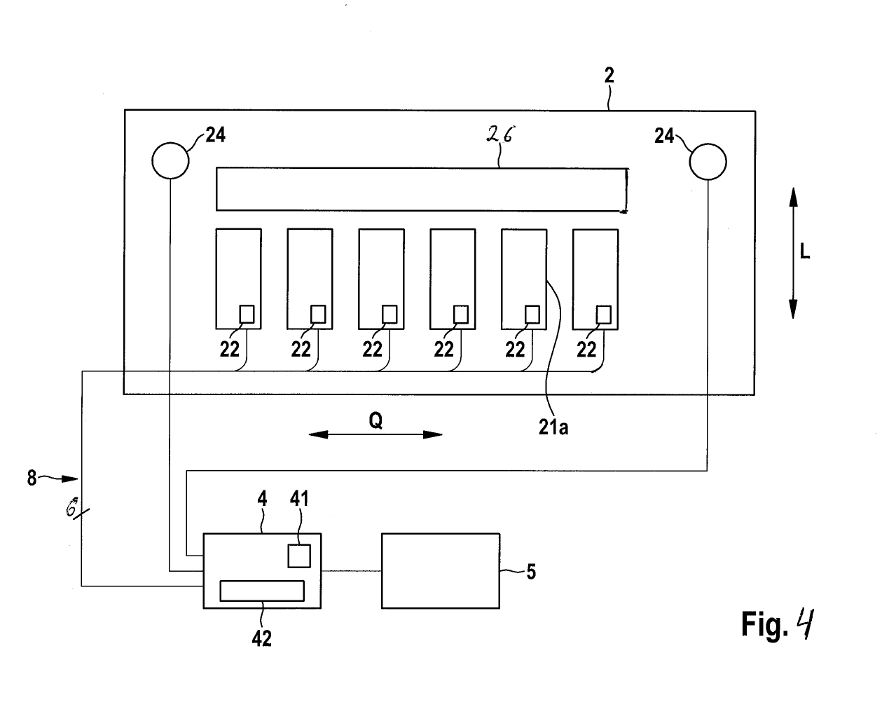

FIG. 4 schematically shows a further embodiment of the system 1 for stopping snoring. The system 1 of FIG. 4 comprises, as described in the previous embodiment, a head support 2, a control unit 4 and an air pump 5.

The arrangement of the first deforming elements 21a in the head support 2 also corresponds in the embodiment of FIG. 4 to an arrangement as it is described in conjunction with the previous embodiments. Instead of the second deforming element as it is described in the previous embodiments, the embodiment of FIG. 4 is provided with a non-active deformable support element 26. That means the height of the support element 26 is given and not variable. The support element can be slightly flexible or can have a flexibility as the first deforming elements 21a so that the head of an individual resting on the head support 2 sinks in uniformly due to its own weight on all sections of the resting area. Furthermore, it can be assured that the individual does not perceive any differences in firmness between the different sections of the resting area depending on whether the section is above the first deforming elements 21a or above the support element 26.

It can be provided that the support element 26 abuts to the row of first deforming elements 21a arranged in cross-direction Q with respect to the longitudinal direction L and has a width in cross-direction Q which substantially corresponds to the width of the first deforming elements 21a arranged in rows. A position change of the head can be achieved merely by appropriate deforming of one or more of the first deforming elements 21a, i.e. by means of an appropriate height change of the one or more of the first deforming elements 21a, i.e. an elevation or a lowering of the height of the section of the head support 2 corresponding to the respective first deforming element 21a. Particularly, a longitudinal tilting (a tilting in longitudinal direction) of the head can be achieved by a simultaneous and preferably uniform lowering or elevating of those deforming elements 21a on which the head rests. The thickness of the support elements 26 in height direction H substantially corresponds to a thickness which is between one fourth and three fourth of the maximum configurable thickness of the first deforming elements 21a, preferably one half of the maximum thickness of the first deforming elements 21a.

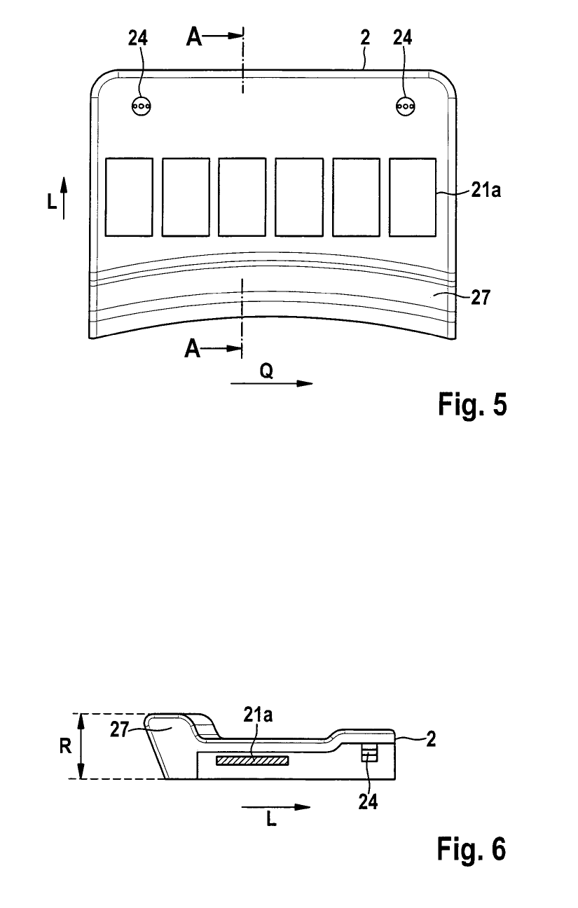

In the top view of FIG. 5, it is schematically shown a further embodiment of the head support 2. As a specific configuration of the support element 26 arranged at the first deforming elements 21a, a neck support element 27 is provided directly at an edge of the head support 2. The neck support element 27 can be arranged directly on the row of the first deforming elements 21a or can be distanced thereto. The neck support element 27 serves as a neck support, if the head of an individual rests on the head support and has a height R (perpendicular to the longitudinal direction L and to the cross-direction Q) which is higher than the corresponding height of the resting area above the first deforming elements 21a on which the head rests. Thereby a function for supporting a neck can be applied, if the head of an individual rests above the first deforming elements 21a and the neck or a side of the neck (in side orientation of the individual) on the neck support element 27. Particularly, in contrast to the thickness of the support element 26 described in conjunction with FIG. 4, the height difference between the resting area of the head above the first deforming elements 21a and the end of the neck support element 27 directed towards the neck of the person is between 2 and 15 cm, preferably between 2 and 10 cm, particularly preferably between 3 and 8 cm, and thus is larger than the maximum thickness of a first deforming element 21a. So a backbone-protecting position of the head on the head support can be assured.

In an embodiment, the row of the first deforming elements 21a and the neck support element 27 can abut.

In a further embodiment, the distance between the row of the first deforming elements 21a and the neck support element 27 can be selected so that when the neck or a side of the neck of an individual rests on the neck support element 27, the backhead rests on the first deforming elements 21a, particularly substantially in the middle with respect to the extension of the first deforming elements 21a into a longitudinal direction L. A deformation or setting of the first deforming elements 21a performed to cause longitudinal tilting, results in the head to tilt over the neck region. This has the advantage that the neck region is not changed in its height, if the first deforming elements 21a are being set.

The neck support element 27 extending along the cross-direction Q can be formed in a slightly curved or convex manner due to ergonomics, wherein the curvature can be concave in the direction of the longitudinal direction L. To limit the length of the first deforming elements 21a in a longitudinal direction with a curved neck support element 27, the arrangement of the first deforming elements 21a can also be curved so that the distance between the neck support element 27 and the row of the first deforming elements 21a can be maintained equal.

In FIG. 6, a cross-sectional view through the head support of FIG. 5 is shown. It can be seen the arrangement of the neck support element 27 which substantially abuts the row of the first deforming elements 21a in longitudinal direction L and which extends along the cross-direction Q of the pillow. The height of the neck support element 27 is higher with respect to the rest of the resting area of the head support 2 to achieve a sufficient support of the neck both in supine orientation and in side orientation.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A head support for stopping a snoring of a sleeping individual, comprising an arrangement of neighboring deforming elements for setting a height of the head support section by section, wherein the deforming elements are arranged to allow a tilting of a head resting on the head support in two directions perpendicular to each other.

2. The head support according to claim 1, wherein a number of first deforming elements neighboring along a row in a cross-direction and one or more support elements are provided, wherein the one or more support elements extend along a longitudinal direction perpendicular to the cross-direction and are offset to the row of the first deforming elements neighboring along the cross-direction.

3. The head support according to claim 2, wherein the one or more support elements abut the first deforming elements neighboring along a cross-direction and are offset with respect to a longitudinal direction perpendicular to the cross-direction.

4. The head support according to claim 2, wherein the one or more support elements are offset to the row of the neighboring first support elements with respect to a longitudinal direction perpendicular to the cross-direction and are distanced therefrom, wherein the distance is selected so that if the neck of the sleeping individual rests on the one or more support elements, the resting area of the back of the head rests on the row of the first deforming elements.

5. The head support according to claim 2, wherein the one or more support elements are arranged on an edge of the head support extending along the cross-direction.

6. The head support according to claim 1, wherein a number of first deforming elements is provided neighboring in a row along a cross-direction, wherein one or more second deforming elements are provided which are offset with respect to a longitudinal direction perpendicular to the cross-direction and which abut the row of the first deforming elements neighbored along the cross-direction.

7. The head support according to claim 1, wherein the deforming elements are configured to be actuated separately to set the height of a section of the head support.

8. The head support according to claim 1, wherein microphones for the detection of a snoring noise are arranged at two opposing sides of the arrangement of the deforming elements.

9. The head support according to claim 1, wherein the deforming elements include chambers for filling with a medium, wherein pressure sensors for determining a pressure in the chamber are arranged in the chambers.

10. A system for stopping a snoring, comprising:
a head support according to claim 1, and
a control unit which is configured to:
detect a snoring noise;
if a snoring noise has been detected, determine an actual orientation of a head resting on the head support, and
actuate the deforming elements so that the head is tilted depending on the detected orientation of the head.

11. The system according to claim 10, wherein the control unit is configured to analyze signal propagation times and/or signal amplitudes of signals detected by microphones and/or a distribution of a pressure signal detected by pressure sensors in the head support.

12. The system according to claim 10, wherein the control unit is configured to perform a learning process to determine at least one preferred tilting movement for the head, wherein, when a snoring occurs, the deforming elements are actuated so that a head resting on the head support is moved according to the preferred tilting movement.

13. The system according to claim 12, wherein the control unit is configured to store the preferred tilting movement in association with a user profile.

14. A method for operating a head support, in particular the head support according to claim 1, comprising the steps of:
detecting whether a snoring noise is present;
if a snoring has been detected, determining an actual orientation of a head resting on the head support; and
actuating the deforming elements so that the head is tilted depending on the detected orientation of the head.
